(19) United States
(12) Patent Application Publication
(10) Pub. No.: US 2023/0277554 A1
(43) Pub. Date: Sep. 7, 2023

(54) COMPOSITION CONTAINING NICKEL-SUPEROXIDE DISMUTASE AND THE LIKE USEFUL FOR HAIRCARE

(71) Applicants: Tzu-Li WANG, Tainan City (TW); Mei-Wen SHIH, Kaohsiung City (TW); Chi-Yao CHEN, Tainan City (TW); Ming-Siang HONG, Tainan City (TW); Matthew LEE, Taipei City (TW)

(72) Inventors: Li-Chi PAN, Taipei City (CN); Tzu-Li WANG, Tainan City (CN); Mei-Wen SHIH, Kaohsiung City (CN); Chi-Yao CHEN, Tainan City (CN); Ming-Siang HONG, Tainan City (CN)

(21) Appl. No.: 18/030,995

(22) PCT Filed: Oct. 8, 2021
(86) PCT No.: PCT/US2021/054075

§ 371 Date: Apr. 8, 2023

Related U.S. Application Data
(60) Provisional application No. 63/089,035, filed on Oct. 8, 2020.

(51) Int. Cl. A61K 31/555; A61K 38/44; A61K 47/10; A61K 9/00; A61P 17/14

(52) U.S. Cl. CPC A61K 31/555; A61K 9/0014; A61K 38/446; A61K 47/10; A61P 17/14

(57) ABSTRACT

Disclosed herein are compositions containing an effective amount of a NiSOD or a derivative thereof useful as a prophylactic treatment for hair graying or follicle degeneration. Also disclosed here are methods for treating or mitigating hair graying and/or follicle degeneration.

### FIELD OF THE INVENTION

This invention pertains to the use of superoxide dismutase (SOD) mimetics, particularly NiSOD compounds and the like in haircare. More particularly, the invention pertains to the prophylactic use of NiSOD compounds and the like for preventing or slowing fair follicle degeneration.

### BACKGROUND OF THE INVENTION

Hair graying and follicle degeneration is a major problem of aging throughout the world. Women and men have all suffered from this condition as we age. Dyeing the hair may address the problem cosmetically but does not treat the underlying issue. Other prior art treatments such as nutritional supplements and coconut oil, ginger, cold laser therapy, and others short of hair transplant all have limited effect.

Therefore, there exists a need for a treatment to prevent or delay hair graying and follicle degeneration.

### SUMMARY OF THE INVENTION

Through trial-and-error, the inventors of the present invention have surprisingly discovered that superoxide dismutase (SOD) mimetics such as nickel-superoxide dismutase (NiSOD) and derivatives thereof have therapeutic properties for reducing or preventing hair follicle degeneration, alopecia, or graying and may be advantageously formulated into haircare products.

Accordingly, one aspect of the present invention provides a haircare composition useful as a prophylactic treatment for hair follicle degeneration, alopecia or hair graying. Compositions in accordance with this aspect of the invention will generally include an effective amount of a SOD mimetic and optionally a surfactant.

SOD mimetics suitable for use in this aspect of the invention is preferrable a NiSOD described in U.S. Pat. No. 9,358,239 (the entire content of which is incorporated herein by reference). NiSOD or derivatives thereof is preferably a six-coordinated derivative having a structural formula:

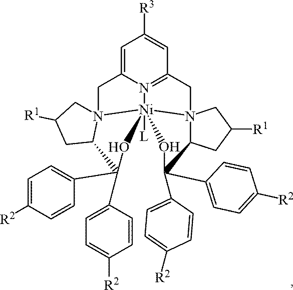

wherein
Wherein R¹ denotes H or OH
Wherein R² denotes H or OMe
Wherein R³ denotes H.

Surfactant is preferably may be an aqueous phase surfactant or an oil phase surfactant suitable for use in a hair product formulation.

Amounts of SOD effective for treating or preventing follicle degeneration, alopecia or hair graying in a subject are generally in the range of from about 2 µg/mL to about 75 µg/mL.

In an exemplary embodiment, the composition includes a stock solution of NiSOD or a derivative thereof having a concentration of from about 0.05 wt% to about 2.0 wt% and from about 1 wt% to about 20 wt% of polyethylene oxide.

polyethylene oxide may be one selected from Polyethylene glycol, Polypropylene glycol, alkoxy polyethylene glycol/Polypropylene glycol copolymer.

In another exemplary embodiment, the composition includes a stock solution of NiSOD or a derivative thereof having a concentration of from about 0.05 wt% to about 2.0 wt%, and about 5 - 18 wt% of a non-ionic surfactant, wherein the non-ionic surfactant is preferably a PEO-PPO copolymer. The PEO-PPO copolymer may be selected from Polyethylene glycol/ Polypropylene glycol copolymer, Triglycerin, Hexaglycerin, PPG-64 _{`} PPG6 _{`} PEG-5 _{`} PEG-6 _{`} PEG-8 _{`} PEG-12 _{`} PEG-14 _{`} PEG-18, and a combination thereof.

In another exemplary embodiment, the composition includes an effective amount of NiSOD or a derivative thereof, and an oil phase surfactant.

In a preferred embodiment, the composition includes a stock solution of NiSOD or a derivative thereof having a concentration from about 0.05 wt% to about 2.0 wt%, and from about 7 wt% to 16 wt% of an oil-soluble Polyol fatty acid ester as the oil phase surfactant.

The oil phase surfactant is preferably select from Propylene Glycol Monostearate, Ethylene glycol fatty acid ester, Sorbitan Monostearate, Propylene Glycol Monolaurate.

In yet another preferred embodiment, the composition may include a stock solution of NiSOD or a derivative thereof having a concentration from about 0.05 wt % to about 2.0 wt% and from about 10% to about 25 wt% of an oil phase PEO-PPO co-polymer nonionic multi-surfactant.

In a preferred embodiment, the oil phase PEO-PPO multi-surfactant may be one selected from Polyoxyethylene sorbitol beeswax and a derivative thereof, Polyoxyethylene oleyl alcohol ether, Polyoxyethylene sorbitol hexastearate, and Polyoxyethylene sorbitol 4.5 oleate.

In a still preferred embodiment, the composition includes a stock solution of NiSOD or a derivative thereof having a concentration from about 0.05 wt% to about 2.0 wt%, from about 0.5% to about 1.5% wtof a GHK-Cu, from about 76.3% to about 84.1% of water, and from about 14.4% to about 22.2% of a polyol.

In still another preferred embodiment, the composition includes a stock solution of a NiSOD or a derivative thereof having a concentration from about 0.05 wt% to about 2.0 wt%, about 0.1% to about 2% of γ -PGA, from about 65.6% to about 89.7% of water, and from about 9.2% to about 33.3% of a polyol.

In a still further embodiment, the composition includes a stock solution of NiSOD or a derivative thereof having a concentration from about 0.05 wt% to about 2.0 wt%, from about 0.1% to about 2% of hyaluronic acid, from about 77.2% to about 89.7% of water, and from about 10.5% to about 21.2% of a poyol.

In another aspect, the present invention also provides a method of treating or mitigating hair follicle degeneration or hair graying. Methods in accordance with this aspect of the invention preferably includes applying a haircare composition as disclosed in this application to the scalp.

Advantageously, when a composition in accordance with the present invention such as a shampoo containing an effective amount of a NiSOD is applied to a subject’s scalp regularly over a period of time, hair graying or follicle degeneration is reduced. Such effect is completely unexpected and only discovered through trial-and-error.

Other aspects and advantages of the invention will be apparent from the following description and the appended claims.

### BRIEF DESCRIPTION OF THE DRAWINGS

FIG. 1 shows an exemplary result of a subject’s reduced hair graying before and after being treated with an exemplary haircare composition of the present invention.

FIG. 2 shows an exemplary dose-dependent antioxidant effect of a NiSOD mimetic.

FIG. 3 shows a comparison of the H₂O₂ inhibitory power of various antioxidant to an exemplary NiSOD.

FIG. 4 shows the ROS scavenging ability of an exemplary NiSOD in cell.

FIG. 5 an exemplary ROS assay of a NiSOD mimetic.

FIG. 6 shows the effect on melanin synthesis in hair of an exemplary NiSOD.

FIG. 7 shows an exemplary before and after comparison of a subject’s hair density treated with an exemplary haircare composition of the present invention.

### DETAILED DESCRIPTION

The unexpected follicle protective effect of superoxide dismutase (SOD) mimetics, as exemplified by NiSOD and its derivatives, was not previously recognized in the art. Although we hypothesize that this surprising discovery might be attributable to SOD mimetics’ antioxidant properties, we disclaim any intention to be bound by any particular theory. The following theoretical discussion is for the purpose of illustration only and shall not be relied upon to limit the scope of the present invention in any way.

Prior art recognizes that SOD has many potential therapeutic potential, including cancer, inflammatory diseases, cystic fibrosis, ischemia, diabetes, rheumatoid arthritis, and certain neurodegenerative diseases. However, where ageing is concerned, the dominant theory postulates that oxygen free-radicals generated in metabolic pathways result in age-related deterioration through oxidative damage to biomolecules, with mitochondria being the main target of attack. Accumulation of oxidative damage is considered to be one of the key mechanisms of aging. Drosophila flies having 75% reduction in SOD activity, showed accelerated loss of olfactory behavior on ageing. While these prior art evidences appear to support the mainstream belief that follicle degeneration is caused by oxidative damage, the fact is that very little is known about the role of SOD in hair loss (alopecia), nor the effect that SOD or its mimetics may have on follicle degeneration.

Although ageing is often associated with alopecia and hair loss, the fact is that age itself is not a determining factor of alopecia and graying. A good percentage of young people may also suffer from these conditions. Prior art further recognizes that alopecia and hair graying may be caused by both intrinsic factors (e.g. genetic and epigenetic mechanisms such as familial premature graying and AGA) and extrinsic factors (e.g. lifestyle choices such as smoking and sun bathing). Experimental evidence seems to support the hypothesis that oxidative stress plays a role in ageing-related hair follicle degeneration. Based on this hypothesis, a number of topical compounds are under investigation, including photoreceptors such as cinnamidpropyltrimonium chloride and solid lipid nanoparticles such as carriers for UV blockers, oral supplementation with 1-cystine and 1-mehionine, and topical melatonin. However, how oxidative stress leads to follicle degeneration is still not completely understood. Thus, SOD as a therapeutic agent for hair loss or hair graying was not previously recognized.

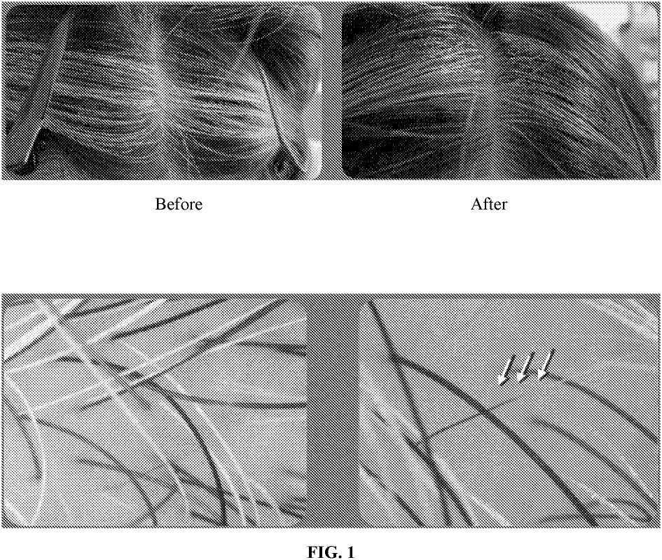

Using NiSOD, applicants have demonstrated that superoxide mimetics may reduce or reverse hair graying and alopecia. FIG. 1 shows photographs of a subject’s hair with gray hair before being treated with a NiSOD composition, and after 30 days of treatment. As shown in the figures, the graying is visibly reduced.

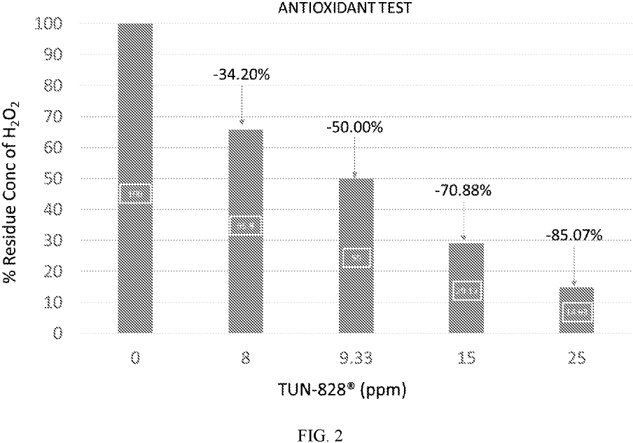

FIG. 2 shows the dose effect of a NiSOD as a scavenger of reactive oxygen species (ROS). As shown in the figure, higher concentrations of NiSOD results in a proportional reduction in ROS. Hydroxyl radicals (HO·) are called reactive oxygen species. These are unstable compounds and react rapidly with most biomolecules without specificity. If it reacts with antioxidants, its absorbance will decrease, so the lower the absorbance, the stronger the ability of antioxidants to capture hydrogen peroxide. Diluted hydrogen peroxide may be used as a standard, provided it is standardized by UV absorption measurement prior to dilution. Hydrogen peroxide solutions can be unstable, so standardization is essential for accurate results. H₂O₂ has a maximum absorbance at 230 nm. To determine the concentration of a Hydrogen Peroxide solution, measure the absorbance at 230 nm. If it reacts with antioxidants, its absorbance will decrease, so the lower its absorbance, the stronger the ability of antioxidants to capture hydrogen peroxide.

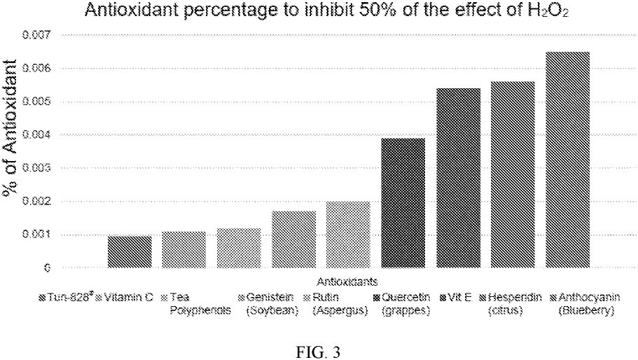

When compared with other common antioxidant, SOD mimetics’ ability to inhibit H₂O₂ is also superior. FIG. 3 shows the percent of antioxidant needed to reach 50% of inhibitory effect on H₂O₂. Referring again to FIG. 2, scavenging capacity of NiSOD on the H₂O₂ was observed to be very effective. NiSOD scavenged H₂O₂ with IC₅₀ value of 9.33 µg/mL, which is even more effective than that of common antioxidant (shown in FIG. 3). The results indicate that NiSOD acts as a very good anti-oxidant agent toward H₂O₂, excellent scavenger of reactive oxygen species and can provide effective protection from oxidative stress.

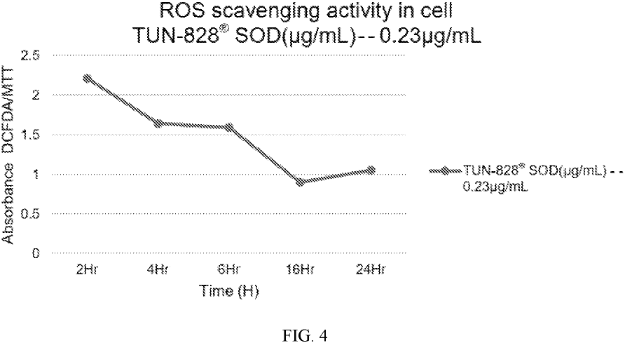

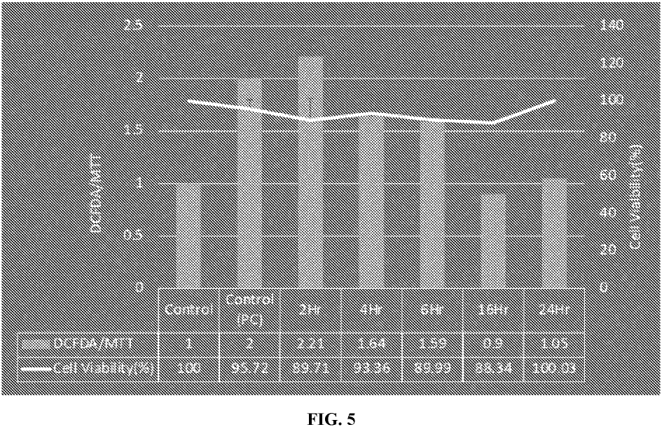

FIG. 4 and FIG. 5 show that after 24 hours, concentration of ROS in cells of subjects treated with NiSOD dropped to the same level as those in healthy control subjects.

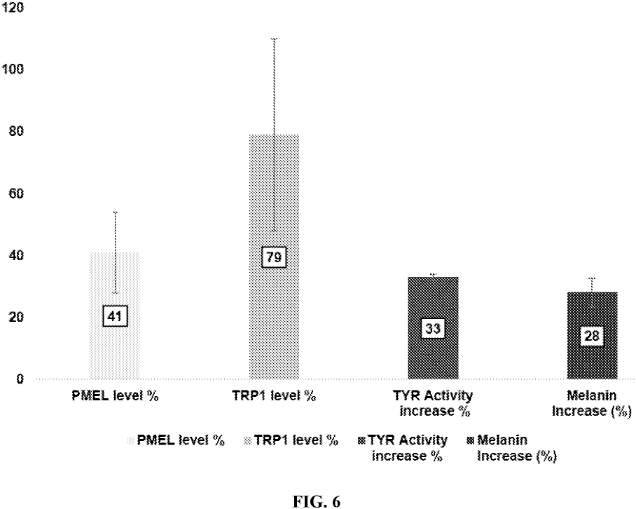

While we do not intend to be bound by any particular theory, NiSOD’s antioxidant capability does appear to support the view that oxidative damage may play a role in hair graying. Yet, given the numerous factors that may cause alopecia and hair graying, NiSOD’s antioxidant property may not be the only reason for its therapeutic effect. We have also unexpected discovered that NiSOD has further effects in malanine synthesis in hair. FIG. 6 shows the effect of an exemplary NiSOD has on melanin synthesis in hair. As shown in the figure, NiSOD treatment resulted in 28% increase in melanin synthesis, 41% increase in pre-melanosomal protein level (PMEL), 79% increase tyrosinase-related protein 1 level (TYRP1) and 33% increase in tyrosinase activity, all of which are melanocyte specific markers.

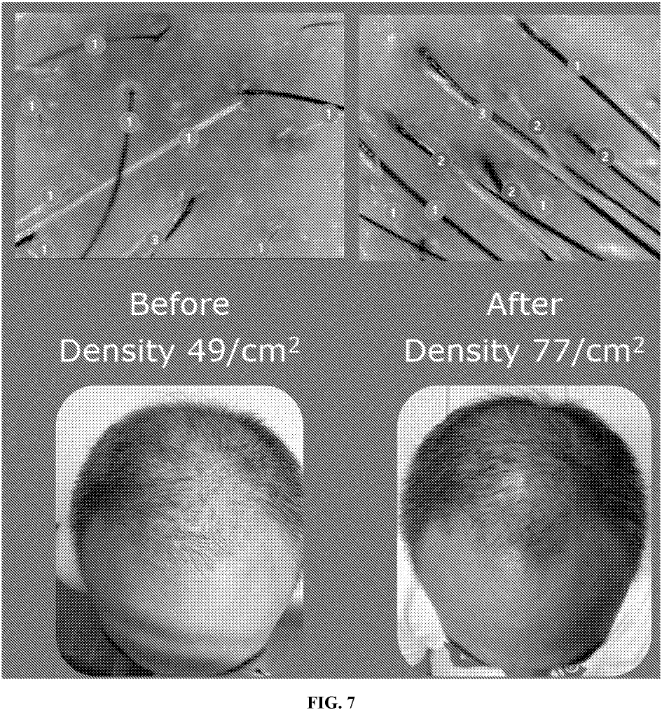

FIG. 7 demonstrates that when a subject suffering from alopecia is treated with a NiSOD composition for 30 days, the degree of hair loss is clearly reduced. The density of hair actually increased from 49/cm² before the treatment to 77/cm² after the 30-day course of treatment, a nearly 2-fold improvement in hair density.

### Haircare Composition and Method of Making

Formulation of haircare products such as shampoo or haircare serum are generally designed to cleanse, condition, and provide a pleasant fresh, clean residual scent to hair. With discovery of the present invention, addition of a SOD mimetic to a haircare formulation such as a shampoo provides the additional benefit of preventing or reducing hair loss and/or hair graying.

Haircare composition in accordance with embodiments of the present invention may be formulated as a shampoo. Exemplary shampoo ingredients may include: water (preferably deionized water, 73 - 90%), primary surfactant (8 - 15% active for foaming and/or lathering), secondary surfactant (2-6% for foam boosting/thickening/mildness improvement), thickening polymers (0 - 2%), salt (0 -2% for thickening), opacifying/pearling agents (0 - 1% active), chelating agent (0.05 - 0.2% for improving hard water performance), conditioning polymers (0 -1% for anti-static, wet/dry conditioning), conditioning emollients (0 - 1%, mostly silicone for wet conditioning), fragrance (0 - 0.5%), and preservatives (typically .001 - 1%)

Surfactants usable in compositions of the present invention may be an oil phase surfactant or an aqueous phase surfactant. Various chemicals are known in the art and are well within the skill in the art.

SOD mimetics are synthetic compounds that mimic the native superoxide dismutase enzyme. They effectively convert the superoxide anion (a reactive oxygen species) into hydrogen peroxide, which may be further converted into water by catalase in vivo. SOD mimetics have been used as a therapeutic agent for cancers, neurodegeneration, inflammatory diseases. Various SOD mimetics are known in the art. The chemical structure of SOD mimetics generally consists of manganese, iron, or copper (and zinc) coordination complexes. Manganese-based SOD mimetics are generally recognized as more therapeutically effective than their counterparts due to their lower toxicity, higher catalytic activity and increased stability in vivo.

It is a surprising discovery that NiSOD and its derivatives show excellent therapeutic and preventive properties in follicle degeneration. Exemplary NiSOD and derivatives may have a structural formula:

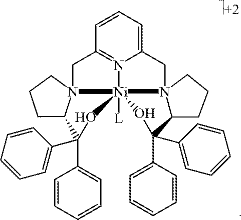

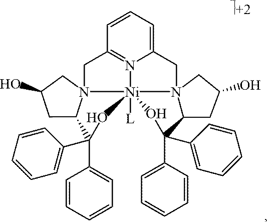

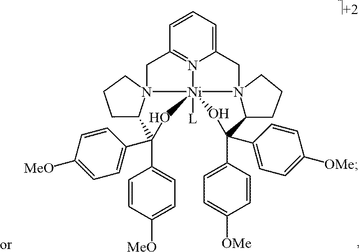

where L is acetonitrile, water or tert-butyl isocyanate. Details of NiSOD suitable for use in a composition of the present invention are as descreibed in U.S. Pat. 9,358,239.

In a preferred embodiment, the SOD mimetic is:

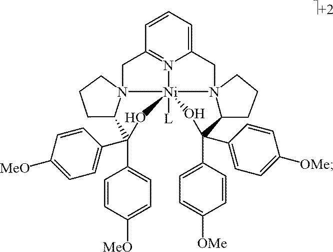

(commercially referred to as TUN-828®).

The following specific embodiments provide further illustration of a haircare composition containing a SOD mimetic:

Embodiment 1

Haircare formulation

| INCI Name | Percentage(%) |
|---|---|
| R.O. Water | 70~80 |
| Polyquaternium-10 | 0.1~1 |
| Polyquaternium-7 | 0.1~1 |
| Cocamidopropyl Betaine | 1~2 |
| Sodium Laureth Sulfate | 8~15 |
| Cocamide MEA | 1~2 |
| Guar Hydroxypropyltrimonium Chloride | 0.1~1 |
| Nickel Bis (Hydroxy Diphenyl MethyIPyrrolidino Methy) Pyridinediy1 t-Butylisocyano Perchlorate(TUN-828®) | 0.5% -1.0% |
| Citric Acid Monohydrate | 0.01~0.1 |
| PhenoxyethanolCaprylyl Glycol | 0.1~0.5 |
| Caprylyl Glycol | 0.1~0.5 |
| Fragrance | 0~0.5 |

Embodiment 2

Skincare formulation

| INCI name | Percentage(%) |
|---|---|
| Aqua | 60~70 |
| Propanediol | 5~10 |
| Glycerin | 5~10 |
| Sorbitol | 1~5 |
| Glycereth-26 | 1~5 |
| Xanthan Gum | 0.05~0.5 |
| PEG-60 Hydrogenated Castor Oil | 0.05~0.5 |
| PEG-40 Hydrogenated Castor Oil | 0.1~0.8 |
| Chlorphenesin | 0.1~0.5 |
| Isopropyl Methylphenol | 0.01-0.1 |
| Disodium Ethylenediaminetetraacetic Acid | 0.01~0.1 |
| Nickel Bis (Hydroxy Diphenyl MethyIPyrrolidino Methy) Pyridinediyl t-Butylisocyano Perchlorate(TUN-828®) | 0.5~2 |
| Perfume | 0.1~0.5 |

### Treatment Method

Patients suffering from hair loss or hair graying may benefit from treatment of a haircare composition of the present invention. When

Within eight months of trying TUN-828® hair tonic, 25.8% of patients had the feeling of scalp health improvement. 48.4% felt their scalp inflammation had improved, 58% felt their hair shedding problems had improved, and 29% felt their black hair density had increased.

Cutibacterium acnes is a troublesome anaerobic pathogen. TUN-828® has the function of forming oxygen internally, so we tried to explore whether such a mechanism can inhibit the growth of Cutibacterium acnes and thereby promote the formation of Staphylococcus epidermidis thus form a micro-ecological environment beneficial to the scalp. The results indicated that TUN-828® have good an efficiency for Cutibacterium acnes inhibition. In 24 hours, TUN-828® can inhibit more than 93% Cutibacterium acnes and more than 99% in 48 hr.

The foregoing description of embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

What is claimed is:

1. A composition useful as a prophylaxis or a therapeutic treatment for hair follicle degeneration, alopecia, or hair graying, comprising:
an effective mount of NiSOD or a derivative thereof, wherein said effective amount is from about 2 ug/mL to about 75 ug/mL.

2. The composition of claim 1, wherein said composition further comprises an aqueous phase surfactant.

3. The composition of claim 2, wherein said composition further comprises:
about 0.05% to about 2.0% of NiSOD stock solution;
about 1% to about 20% of polyoxyethane series or polyethylene oxide [symbol] .

4. The composition of claim 3, wherein said polyoxyethane include Polyethylene glycol, Polypropylene glycol, alkoxy polyethylene glycol/Polypropylene glycol copolymer.

5. The composition of claim 2, further comprises:
about 0.05% to about 2.0% of NiSOD stock solution;
about 5% to about 18% of Surfactant polyoxyethylene-polyoxypropylene series nonionic surfactant.

6. The composition of claim 5, wherein said Surfactant polyoxyethylene-polyoxypropylene series nonionic surfactant include Polyethylene glycol/ Polypropylene glycol copolymer, Triglycerin, Hexaglycerin, PPG-64 · PPG6 · PEG-5 · PEG-6 · PEG-8 · PEG-12 · PEG-14 · PEG-18, and a combination thereof.

7. The composition of claim 1, wherein said composition is one suitable for applying to hair and further comprising an oil phase surfactant.

8. The composition of claim 7, wherein said composition further comprises:
about 0.05% to about 2.0% of NiSOD stock solution;
about 7% to about 16% oil-soluble Polyol fatty acid ester.

9. The composition of claim 8, wherein said Oil-soluble polyol fatty acid ester include Propylene Glycol Monostearate, Ethylene glycol fatty acid ester, Sorbitan Monostearate, Propylene Glycol Monolaurate.

10. The composition of claim 7, wherein said composition further comprises:
about 0.05% to about 2.0% of NiSOD stock solution;
about 10% to about 25% Multi-surfactant polyoxyethylene-polyoxypropylene series nonionic surfactant.

11. The composition of claim 10, wherein said Oil-soluble surfactant polyoxyethylene-polyoxypropylene series nonionic surfactant include Polyoxyethylene sorbitol beeswax and a derivative thereof, Polyoxyethylene oleyl alcohol ether, Polyoxyethylene sorbitol hexastearate, and Polyoxyethylene sorbitol 4.5 oleate.

12. The composition of claim 1, wherein said composition further comprises:
about 0.05% to about 2.0% of NiSOD stock solution;
about 0.5% to about 1.5% of Cu-peptide;
about 76.3% to about 84.1% water; and
about 14.4% to about 22.2% polyol.

13. The composition of claim 1, wherein said composition further comprises:
about 0.05% to about 2.0% of NiSOD stock solution;
about 0.1% to about 2% of γ-PGA;
bout 65.6% to about 89.7% of water; and
about 9.2% to about 33.3% polyol.

14. The composition of claim 1, wherein said composition further comprises:
about 0.05% to about 2.0% of NiSOD stock solution;
about 0.1% to about 2% of Hyaluronic acid;
about 77.2% to about 89.7% of water; and
about 10.5 to about 21.2% of polyol.
